(19) United States
(12) Patent Application Publication
(10) Pub. No.: US 2022/0409681 A1
(43) Pub. Date: Dec. 29, 2022

(54) LACTOBACILLUS SALIVARIUS LS97 AND APPLICATION THEREOF

(71) Applicant: WECARE PROBIOTICS CO., LTD., Jiangsu (CN)

(72) Inventors: Shuguang FANG, Jiangsu (CN); Keke CHEN, Jiangsu (CN); Jianguo ZHU, Jiangsu (CN); Chun LIU, Jiangsu (CN); Yingying ZHAN, Jiangsu (CN)

(21) Appl. No.: 17/777,347

(22) PCT Filed: Jul. 2, 2020
(86) PCT No.: PCT/CN20/99923

§ 371 Date: May 17, 2022

(30) Foreign Application Priority Data
Nov. 25, 2019 (CN) 201911163718.1

(51) Int. Cl. A61K 35/747; C12N 1/20; A61P 1/02

(52) U.S. Cl. CPC A61K 35/747; C12N 1/205; A61P 1/02; C12R 2001/225

(57) ABSTRACT

Disclosed is a Lactobacillus salivarius LS97 and an application thereof. The strain was deposited in the China General Microbiological Culture Collection Center on Dec. 10, 2018 with a deposit number of CGMCC NO.16922 and a classification name of Lactobacillus salivarius, the deposit address being China General Microbiological Culture Collection Center, Institute of Microbiology, Chinese Academy of Sciences, No. 1 West Beichen Road, Chaoyang District, Beijing 100101, China. The Lactobacillus salivarius LS97 can effectively inhibit the growth of Streptococcus sobrinus.

### TECHNICAL FIELD

The present application relates to the field of microorganisms, and particularly to a Lactobacillus salivarius LS97 and an application thereof.

### BACKGROUND

Streptococcus sobrinus was once called as “Streptococcus Distant” or “Streptococcus Fuzzy”, which got its name because of its fuzzy coat under the electron microscope observation. For a long time, Streptococcus sobrinus has been considered as an important kind of cariogenic bacteria. Compared with Streptococcus mutans, Streptococcus sobrinus has a higher acid-producing capability and higher acid resistance, and has more close relation with heavy caries, thus attracting attention from researchers increasingly.

At present, the methods of preventing and treating dental caries caused by Streptococcus sobrinus mainly include drug method, dietary-controlling method, mechanical removal method and the like, and for example, by applying compounds such as fluorides, phenolic compounds or strong oxidizers, the aim of killing Streptococcus sobrinus could be achieved. However, drugs overuse is prone to cause an increase of drug resistance in bacteria and also accompanied by corresponding side effects. The dietary-controlling is difficult to achieve due to problems such as the complex composition of modern diets (including many natural sugars, refined sugars or sugar substitutes, etc.). Herein, it has become a research focus to develop new solutions for prevention and treatment.

“Probiotics” is a collective term covering a class of active microorganisms that are beneficial to the host, whose application in intestinal health has been widely studied. In the aspect of antagonizing pathogenic bacteria, probiotics can achieve the purpose of inhibiting pathogenic bacteria by producing bacteriocins, hydrogen peroxide, organic acids or other ways. Taking Streptococcus mutans as an example, a Lactobacillus plantarum CCFM8724 strain was screened out by Chen et al. (China Publication No. CN109908185A) which can effectively inhibit the biofilm formation of Streptococcus mutans, reducing the production of Streptococcus mutans exopolysaccharide by about 80%. A Lactobacillus rhamnosus LR863 strain was screened out by Zhao et al. (China Publication No. CN108048347A), which had an inhibition zone diameter of 15.35±0.06 mm for Streptococcus mutans. Nevertheless, it should be pointed out that although there are lots of reports on the selection of strains that can antagonize the dental caries bacteria, Streptococcus mutans, there are few reports on the screening of strains that can effectively inhibit Streptococcus sobrinus, and the relevant strain resources are almost blank.

### SUMMARY

Provided in the present application is Lactobacillus salivarius LS97, which can effectively inhibit the growth of Streptococcus sobrinus, and an application thereof.

A first aspect of the present application provides a Lactobacillus salivarius, named LS97, which has been deposited in the China General Microbiological Culture Collection Center on December 10, 2018 with a deposit number of CGMCC NO.16922, whose classification name is Lactobacillus salivarius and deposition address is the Institute of Microbiology of Chinese Academy of Sciences, No. 3, Yard 1, Beichen West Road, Chaoyang District, Beijing.

A second aspect of the present application provides an application of Lactobacillus salivarius LS97 in the preparation of a drug for alleviating dental caries.

Furthermore, the drug includes a living strain, a dried strain or a metabolite of Lactobacillus salivarius LS97.

Furthermore, the drug includes a pharmaceutically acceptable carrier.

Furthermore, a dosage form of the drug is a tablet, capsule, oral liquid or lyophilized powder.

In view of the above solution, the present application has the following benefits at least.

1. Lactobacillus salivarius LS97 can inhibit the growth and reproduction of Streptococcus sobrinus; additionally, it has good co-aggregation capability with planktonic Streptococcus sobrinus, and can eliminate Streptococcus sobrinus through antagonism, co-aggregation effect and the like.

2. Through investigating the capability of Lactobacillus salivarius LS97 to inhibit other pathogenic bacteria, it is found that LS97 can effectively inhibit Streptococcus mutans and Streptococcus sanguinis.

3. The acid-producing and lysozyme tolerance of the strain is evaluated using in vitro experiments, and LS97 can tolerate a concentration up to 1 mg/mL of lysozyme and has an acid-producing capability much lower than that of Streptococcus sobrinus.

4. It is shown in the rat experiment results that Lactobacillus salivarius LS97 can effectively alleviate the development of dental caries, with better effect than chlorhexidine.

The above description is merely a summary of the technical solution in the present application. For a better understanding of the technical means of the present application, and for enabling an implement according to the description contents, the present disclosure is described in detail below by the preferred embodiments of the present application hereinafter.

### BRIEF DESCRIPTION OF DRAWINGS

FIG. 1 is the graph showing a comparison between self-aggregation property of different Lactobacillus strains and co-aggregation property of different Lactobacillus strains with Streptococcus sobrinus in the present application;

FIG. 2 is the graph showing a comparison of acid-producing property during the growth process of different Lactobacillus strains in the present application;

FIG. 3 is the graph showing a result of the inhibition capability of Lactobacillus salivarius LS97 on the growth of Streptococcus mutans and Streptococcus sanguinis in the present application;

FIG. 4 is the graph showing a comparison of rat dental caries score in the present application.

### DETAILED DESCRIPTION

The specific embodiments of the present application will be further described in detail below with reference to the examples. Although the present application is illustrated through the following examples, the scope of the present application is not limited to the examples.

### EXAMPLE 1

### Selection of Lactobacillus salivarius LS97

1) Strain Screening

The 5 mL diluent of digestive tract fluid, which was collected from healthy infants from Bama Town, Hechi City, Guangxi Zhuang Autonomous Region, China (before the collection, either of the population had no antibiotics administration, no history of taking probiotics, and no history of gastrointestinal disease), was taken and diluted to 10⁻⁶ through 10-fold serial dilutions. Dilutions of various concentrations were taken out 200 μL each and were spread respectively on the selective MRS medium. After anaerobic culture at 37° C. for 48 h to 72 h, different unit colonies on the plate were separated and obtained. The aforementioned different unit colonies were picked by a sterilized inoculating loop, were purified by lining separation on the MRS solid medium, and anaerobically cultured at 37° C. for 48 h so that a total of 11 pure Lactobacillus strains were obtained, named A1-A11 respectively.

2) Test of the Isolated Strain Antagonizing the Streptococcus sobrinus

By the double-layer cylinder-plate method, the inhibition effect of the isolated strains A1-A11 on the growth of Streptococcus sobrinus strain (ATCC27352, purchased from ATCC) was investigated. 1.5% (w/v) BHI agar medium was poured into a plate, and after solidification, a sterile Oxford cup was fixed in the appropriate place, and then a mixture of 0.7% (w/v) BHI medium and Streptococcus sobrinus (10⁷ CFU/mL) was poured onto the lower medium. After solidification, the Oxford cup was taken out, and each cavity was added into 100 μL of Lactobacillus culture supernatant, and the aforementioned plate was placed at 37° C. (5% CO₂) circumstance for culturing overnight.

The experimental result is shown in Table 1. Except for A1, A3, A7 and A11, the other Lactobacillus could effectively inhibit the growth of Streptococcus sobrinus, among which Lactobacillus A5 had the best inhibition capability and its inhibition zone with a diameter reaching 23.1±1.1 mm, possessing the inhibition capability higher than other strains on Streptococcus sobrinus.

TABLE 1
Investigation into the inhibition capability of different
Lactobacillus on Streptococcus sobrinus

| Strain | Diameter of inhibition zone (mm) |
|---|---|
| A1 | ND |
| A2 | 10.9 ± 0.6 |
| A3 | ND |
| A4 | 13.2 ± 1.0 |
| A5 | 23.1 ± 1.1 |
| A6 | 16.8 ± 0.9 |
| A7 | ND |
| A8 | 14.3 ± 1.1 |
| A9 | 15.9 ± 1.3 |
| A10 | 9.4 ± 0.5 |
| A11 | ND |

Note:
ND refers to that no inhibition zone was detected.

3) Analysis of Self-Aggregation and Co-Aggregation Property of Isolated Strain

The different strains obtained overnight under anaerobic condition and Streptococcus sobrinus, were centrifuged at 3000 r/min for 10 min, washed twice with PBS (10 mmol/L, pH 7.2), resuspended, and adjusted to a bacterial concentration with OD₆₀₀ of 0.5. In the co-aggregation experiment, Streptococcus sobrinus and the different strains were each taken out 500 μL and detected for the initial OD₆₀₀ values Ax₀ and Ay₀ respectively, and then were mixed and placed anaerobically at 37° C. for 24 hours, then measured the OD₆₀₀ value A₂₄. The co-aggregation capability=[(Ax₀+Ay₀)−2×A₂₄)]/(Ax₀+Ay₀)×100. In the self-aggregation experiment, the different Lactobacillus suspensions were taken out 1000 μL respectively, and detected for the initial OD₆₀₀ value A₀, and after placed anaerobically at 37° C. for 24 hours, detected for the OD₆₀₀ value A₂₄. The self-aggregation capability=(A₀−A₂₄)/A₂₄×100.

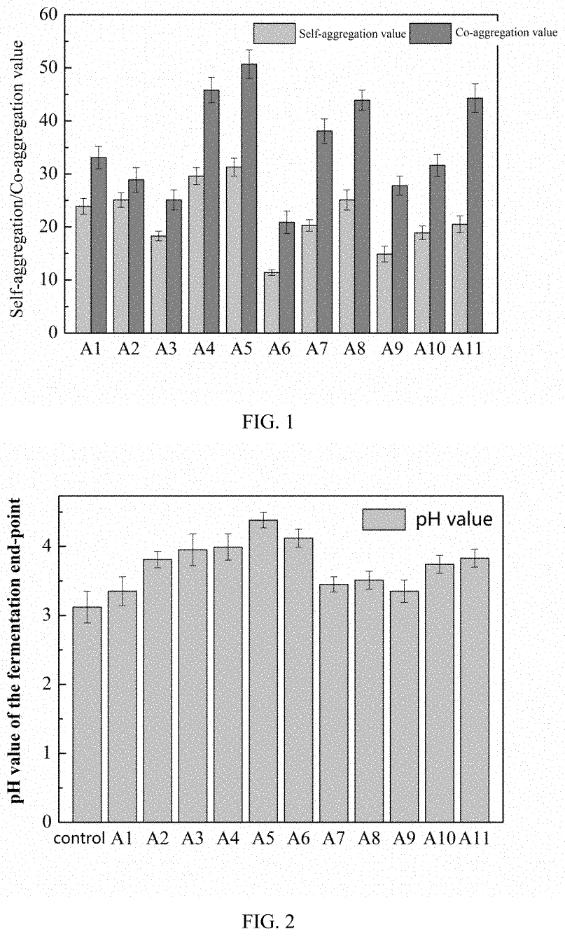

The result is shown in FIG. 1. Lactobacillus A5 had excellent self-aggregation and co-aggregation capability, whose self-aggregation and co-aggregation capability were much higher than those of other strains, indicating that Lactobacillus A5 could also eliminate Streptococcus sobrinus by co-aggregation and other effects.

4) Analysis of Lysozyme Tolerance of Isolated Strain

The lysozyme tolerance of the strain was investigated by the double-layer cylinder-plate method, of which the specific operations were as follows: 1.5% (w/v) agar medium was used to prepare the lower medium, and after solidification, a sterile Oxford cup was fixed in the appropriate place, 100 μL probiotics suspension with OD₆₀₀ adjusted to 0.5 was taken out, added to MRS medium containing 0.8% (W/V) agar cooled to about 50° C., mixed uniformly and poured onto a plate. After solidification, the Oxford cup was taken out, and 50 μL of lysozyme solutions with different concentrations (0.2 mg/mL, 0.4 mg/mL, 0.6 mg/mL, 0.8 mg/mL, 1.0 mg/mL and 10.0 mg/mL, respectively) were added into the cavities, and the diameter of inhibition zone was measured after anaerobic culture at 37° C. for 48 h.

The experimental result is shown in Table 2. Except for strain A1, all other strains could grow normally in 1 mg/mL lysozyme solution, among which strain A7 could tolerate a lysozyme concentration of 10 mg/mL. Under normal circumstances, the concentration of lysozyme contained in the oral cavity is 1 μg/mL to 57 μg/mL. The above results show that the tolerance of aforementioned strains to lysozyme greatly exceeded the normal lysozyme concentration in the oral cavity.

TABLE 2
Investigation into the inhibition property of different
lysozyme concentrations on strains

| Diameter | | | | | | | |
|---|---|---|---|---|---|---|---|
| of | | | | | | | |
| inhibition | | | | | | | |
| zone | Lysozyme concentration ( mg/mL ) | | | | | | |
| (mm) | 0 | 0.2 | 0.4 | 0.6 | 0.8 | 1 | 10 |
| A1 | 0 | 0 | 0 | 0 | 0 | 9.7 ± 0.9 | 15.9 ± 1.6 |
| A2 | 0 | 0 | 0 | 0 | 0 | 0 | 10.5 ± 1.0 |
| A3 | 0 | 0 | 0 | 0 | 0 | 0 | 9.6 ± 1.0 |
| A4 | 0 | 0 | 0 | 0 | 0 | 0 | 10.9 ± 1.2 |
| A5 | 0 | 0 | 0 | 0 | 0 | 0 | 12.1 ± 1.1 |
| A6 | 0 | 0 | 0 | 0 | 0 | 0 | 11.3 ± 0.8 |
| A7 | 0 | 0 | 0 | 0 | 0 | 0 | 0 |
| A8 | 0 | 0 | 0 | 0 | 0 | 0 | 12.1 ± 1.1 |
| A9 | 0 | 0 | 0 | 0 | 0 | 0 | 9.3 ± 0.7 |
| A10 | 0 | 0 | 0 | 0 | 0 | 0 | 9.7 ± 1.2 |
| A11 | 0 | 0 | 0 | 0 | 0 | 0 | 11.3 ± 1.1 |

5) Analysis of Acid-Producing Property of Isolated Strain

Bacterial suspensions of different Lactobacillus strains and Streptococcus sobrinus were detected for the absorbance, adjusted to OD600 value of 0.5±0.02 with phosphate buffer, inoculated into MRS or BHI medium according to a 2% (v/v) inoculation dosage respectively, cultured at 37° C. (5% CO₂) for 24 h, and detected for the pH value in the culture at the end point. The result is shown in FIG. 2. The cariogenic bacteria, Streptococcus sobrinus, had a high acid-producing property and the lowest pH at the determination end point. By comparison, the acid-producing property of strains A5 and A6 was relatively weak. Probiotics can produce organic acids after fermentation and reduce the pH value of the environment. Although such property may inhibit the reproduction of some pathogenic bacteria to a certain extent, in the oral environment, the production of organic acids, especially lactic acid, is also prone to cause greater risk of dental caries. Therefore, in view of acid-producing property, the strains with relatively weak acid-producing property are more conducive to the application of oral protection.

By summarizing the results of the above different screening schemes, based on the high inhibition capability on Streptococcus sobrinus and high self-aggregation and co-aggregation property, and through combining the analysis of the lysozyme tolerance and the acid-producing property of the isolated strain, strain A5 was screened and obtained.

### EXAMPLE 2

### Identifying the Strain

For the strain obtained by Example 1, the identification method is as follows.

To the obtained strain A5, 16s rDNA sequencing identification and physiological and biochemical identification were applied, in which the universal primer sequences of 16s rDNA included: 27F as: 5′-AGAGTTTGATCCTGGCTCAG-3′ (Seq ID No: 1), and 1492R as: 5′-GGTTACCTTGTTACGACTT-3′ (Seq ID No: 2). The 16S rDNA gene sequence of strain A5 was amplified and sequenced, and the PCR amplification product was sent to Shanghai Sangon Biotech. (Shanghai) Co., Ltd. for sequencing. The nucleotide sequence of 16S rDNA of strain A5 is shown in Seq ID No: 3. By 16s rDNA gene comparison, the strain had a similarity rate reaching 100% when compared with Lactobacillus salivarius strain in Genebank. Combining the result of physiological and biochemical identification, strain A5 was identified as a Lactobacillus salivarius, named Lactobacillus salivarius LS97, of which the physiological and biochemical properties are shown in Table 3.

TABLE 3
Physiological and biochemical identification result of strain A5

| Biochemical | Identification | Biochemical | Identification |
|---|---|---|---|
| test item | result | test item | result |
| Salicin | − | Mannose | − |
| Maltose | + | Gluconate | − |
| Amygdalin _ | − | pH 3.5 | + |
| Galactose | + | Gelatin | − |
| | | Liquefaction | |
| Arabinose | − | Anaerobic growth | + |
| Mannose | − | 15° C. growth | − |
| Fructose | + | Ammonia | − |
| | | production from | |
| | | arginine | |

Note:
+: positive result; −: negative result

EXAMPLE 3

### Analysis of the Inhibition Capability of Lactobacillus salivarius LS97 on the Growth of Streptococcus mutans and Streptococcus sanguinis

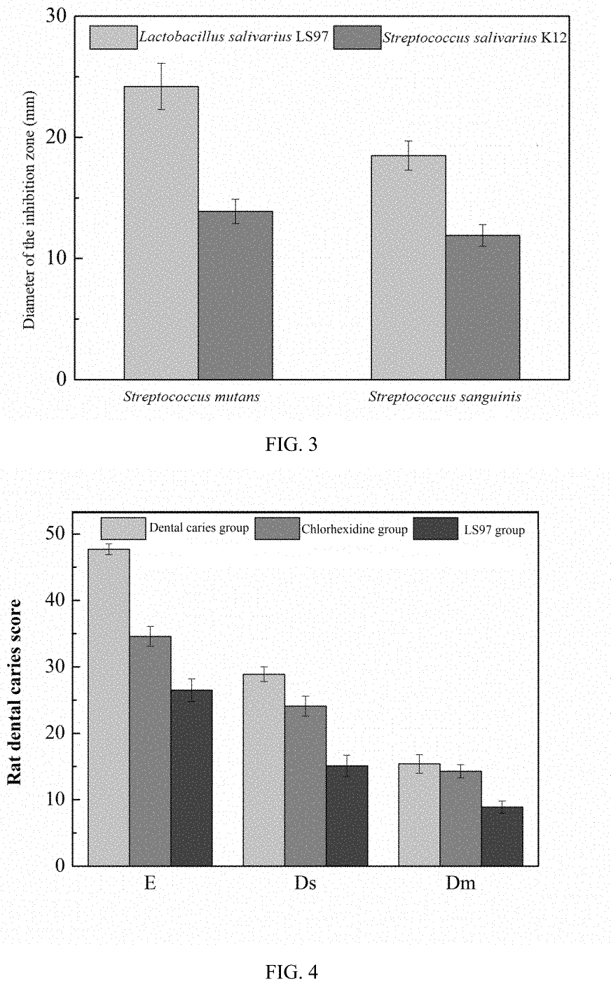

The double-layer cylinder-plate method was also used to investigate the growth inhibition of Lactobacillus salivarius LS97 on Streptococcus mutans (UA159, purchased from ATCC) and Streptococcus sanguinis (SK36, purchased from ATCC). 1.5% (w/v) agar medium was poured into a plate, and after solidification, a sterile Oxford cup was fixed in the appropriate place, and then a mixture of 0.7% (w/v) BHI medium and Streptococcus mutans (10⁷ CFU/mL), or a mixture of 0.7% (w/v) BHI medium and Streptococcus sanguinis (10⁷ CFU/mL), was poured onto the lower medium. After solidification, the Oxford cup was taken out, and each cavity was added into 100 μL of Lactobacillus culture supernatant. The aforementioned plate was placed at 37° C. (5% CO₂) circumstance for culturing overnight, and Streptococcus salivarius (K12, purchased from ATCC) reported previously with the capability to inhibit the above two strains was used as a control strain. The experimental result is shown in FIG. 3. Lactobacillus salivarius LS97 could effectively inhibit the growth of Streptococcus mutans and Streptococcus sanguinis with the inhibition zone of a diameter reaching 24.2±1.9 mm and 18.5±1.2 mm respectively, and had a higher inhibition capability on the two aforementioned cariogenic bacteria than the capability of the control strain, Streptococcus salivarius K12.

### EXAMPLE 4

### Analysis of the Alleviating Effect of Lactobacillus salivarius LS97 on Dental Caries in Rats

SPF female Wistar rats (about 3 weeks old) were obtained and housed in a barrier environment with constant temperature and humidity, with 12 h of light and 12 h of darkness. All rats were acclimated to the environment and diet in a week before experiment, and then were randomly divided into groups according to body weight. The rats were randomly divided into 4 groups according to body weight with 10 rats in each group, among which one group was set as the blank control group (blank group), one group was set as the dental caries model group (dental caries group), and two groups were set as the intervention group. The intervention groups were wiped S. sobrinus continuously for 7 days, and after confirming that S. sobrinus was successfully colonized in the rat's oral cavity, the corresponding experimental groups were wiped chlorhexidine (chlorhexidine group) and LS97 (LS97 group) continuously for 7 days respectively, and then 3 times a week. Chlorhexidine, an ionic drug with good antibacterial effect, was used as a positive control group. The cariogenic bacteria S. sobrinus and LS97 were both cultured overnight for 12 h. After adjusted to a bacterial concentration of 1×10⁸ CFU/mL, 0.2 mL of bacterial suspension was wiped onto the rat molars by sterile cotton swab, and namely, the cotton swab that absorbed bacterial suspension sufficiently worked on each quarter of the rat's oral teeth for the same 15 s. During the experiment, except for the blank control group fed normal feed and drinking water, other groups were all fed cariogenic feed Diet 2000# and drinking water added with 5% sucrose, in which the main components of cariogenic feed are shown in Table 4. The experimental period was maintained for 10 weeks.

TABLE 4
Main components of cariogenic feed Diet 2000#

| Component | Weight percentage (%) |
|---|---|
| Whole wheat flour | 6 |
| Sucrose | 56 |
| Refined milk powder | 28 |
| Alfalfa leaf powder | 3 |
| Dehydrated dry powder | 1 |
| Yeast | 4 |
| Salt | 2 |

1) The effect of Lactobacillus salivarius LS97 on the body weight of rats

The result of body weight measurement of rats before and after experiment is shown in Table 5. During the rat experiment, there was no significant difference in body weight increment between the experimental group and control group (P>0.05), indicating that all experimental animals showed good physical condition during the animal experiment.

TABLE 5
Result of body weight measurement before and after the rat experiment

| | Body weight of | Body weight of | |
|---|---|---|---|
| | rats before the | rats after the | Rat body weight |
| Group | experiment (g) | experiment (g) | increment (g) |
| Blank group | 64.03 ± 3.15 | 205.94 ± 21.35 | 140.42 ± 23.74 |
| Dental caries | 62.98 ± 3.56 | 209.32 ± 20.12 | 145.12 ± 21.75 |
| group | | | |
| Chlorhexidine | 60.32 ± 5.28 | 212.49 ± 23.86 | 147.83 ± 19.73 |
| group | | | |
| LS97 group | 60.13 ± 6.03 | 211.51 ± 19.75 | 147.09 ± 19.94 |

Note:
there was no significant difference in body weight among all the groups in each column (P > 0.05).

2) Changes of Streptococcus sobrinus (S. sobrinus) in the Oral Cavity of Rats

At the 3rd, 7th, and 10th weeks of the experiment (corresponding to the early, middle and late stages of the experiment, respectively), the first, second and third samplings were performed on the oral teeth of rats, and samples were spread on MRS solid medium added with vancomycin of 12 μg/mL for Streptococcus sobrinus count. Streptococcus sobrinus in the oral cavity of rats that belonged to the dental caries model group and the intervention group, after infected with Streptococcus sobrinus, was in the range of 2-4×10⁵ CFU/mL, and there was no significant difference among the groups, as revealed in Table 6 showing the count result of Streptococcus sobrinus in the first time Sampling. Since the experimental group was intervened, the number of Streptococcus sobrinus decreased by 1 to 2 orders of magnitude in the two groups, in which the number of Streptococcus sobrinus decreased more significantly in the Lactobacillus salivarius LS97 group. Additionally, the number of Streptococcus sobrinus showed an increment circumstance in the chlorhexidine group during the third test, presumably resulting from that the strain had developed drug resistance. The above results showed that both chlorhexidine and Lactobacillus salivarius LS97 could inhibit the number of Streptococcus sobrinus, yet the intervention effect of Lactobacillus salivarius LS97 was better than that of the chlorhexidine group.

TABLE 6
Count situation of Streptococcus sobrinus in oral sampling of
experimental rats in each group in different stages (Log value)

| Sampling | Blank | Dental caries | Chlorhexidine | |
|---|---|---|---|---|
| number | group | group | group | LS97 group |
| 1 | <30 | 5.41 ± 0.12 | 5.53 ± 0.23 | 5.51 ± 0.21 |
| 2 | <30 | 5.28 ± 0.09 | 4.00 ± 0.19 | 3.63 ± 0.13 |
| 3 | <30 | 5.17 ± 0.11 | 4.31 ± 0.13 | 3.48 ± 0.12 |

3) Rat Dental Caries Score

After the rat experiment was finished, the rats were killed and the rat molars were taken out. The molars were placed in a 0.4% murexide dye liquor and soaked for 12 h overnight for dyeing. After the molars were rinsed by clean water and dried naturally, the caries lesion condition of rat molars in each group was observed under a stereo microscope. Caries scoring was performed on the rat teeth by using the Keyes caries scoring method. According to Keyes' classic method for evaluating dental caries, the lesion degree of dental caries is divided into four grades: E-grade caries, caries lesion involving enamel only; Ds-grade caries, enamel caries lesion and caries lesion involving the outer dentin less than ¼; Dm-grade caries, caries lesion involving the dentin thickness within ¼ to ¾; Dx-grade caries, caries lesion involving the dentin thickness more than ¾, and the result is shown in FIG. 4.

It can be seen from FIG. 4 that compared with the dental caries model group, both chlorhexidine group and LS97 group could effectively reduce the development of molar enamel caries (E), dentin superficial caries (Ds) and dentin middle caries (Dm) in rats. In addition, comparing the effect of chlorhexidine group and LS97 group, it could be seen that chlorhexidine group effectively reduced E caries and Ds caries, yet its effect on Dm caries was not obvious. By comparison, the effect of LS97 group on reducing the development of dental caries was higher than that of chlorhexidine, indicating that the effect of Lactobacillus salivarius LS97 was better than that of chlorhexidine in the treatment of dental caries.

The above descriptions are only the preferred embodiments of the present application and are not intended to limit the present application. It should be pointed out that for those skilled in the art, without departing from the technical principles of the present application, any improvement and modification may be made, and those improvements and modification should also be regarded as falling within the protection scope of the present application.

1. A Lactobacillus salivarius LS97, named LS97, which has been deposited in the China General Microbiological Culture Collection Center on Dec. 10, 2018 with a deposit number of CGMCC NO.16922.

2-5. (canceled)

6. A method for alleviating dental caries, comprising administering an effective amount of a drug prepared from the Lactobacillus salivarius LS97 according to claim 1 to subject in need thereof.

7. The method according to claim 6, wherein the drug comprises a living strain, a dried strain or a metabolite of Lactobacillus salivarius LS97.

8. The method according to claim 6, wherein the drug comprises a pharmaceutically acceptable carrier.

9. The method according to claim 6, wherein a dosage form of the drug is a tablet, capsule, oral liquid or lyophilized powder.


SEQUENCE LISTING (3 sequences)

SEQ ID NO: 1 - DNA, 20 bp, Artificial sequence
  primer
agagtttgatcctggctcag

SEQ ID NO: 2 - DNA, 19 bp, Artificial sequence
  primer
ggttaccttgttacgactt

SEQ ID NO: 3 - DNA, 1505 bp, Lactobacillus salivarius
tggctcaggacgaacgctggcggcgtgcctaatacatgcaagtcgaacgaaactttcttacaccgaatgcttgcattcaccgtaagaagttgagtggcggacgggtgagtaacacgtgggtaacctgcctaaaagaaggggataacacttggaaacaggtgctaataccgtatatctctaaggatcgcatgatccttagatgaaagatggttctgctatcgcttttagatggacccgcggcgtattaactagttggtggggtaacggcctaccaaggtgatgatacgtagccgaactgagaggttgatcggccacattgggactgagacacggcccaaactcctacgggaggcagcagtagggaatcttccacaatggacgcaagtctgatggagcaacgccgcgtgagtgaagaaggtcttcggatcgtaaaactctgttgttagagaagaacacgagtgagagtaactgttcattcgatgacggtatctaaccagcaagtcacggctaactacgtgccagcagccgcggtaatacgtaggtggcaagcgttgtccggatttattgggcgtaaagggaacgcaggcggtcttttaagtctgatgtgaaagccttcggcttaaccggagtagtgcattggaaactggaagacttgagtgcagaagaggagagtggaactccatgtgtagcggtgaaatgcgtagatatatggaagaacaccagtggcgaaagcggctctctggtctgtaactgacgctgaggttcgaaagcgtgggtagcaaacaggattagataccctggtagtccacgccgtaaacgatgaatgctaggtgttggagggtttccgcccttcagtgccgcagctaacgcaataagcattccgcctggggagtacgaccgcaaggttgaaactcaaaggaattgacgggggcccgcacaagcggtggagcatgtggtttaattcgaagcaacgcgaagaaccttaccaggtcttgacatcctttgaccacctaagagattaggctttcccttcggggacaaagtgacaggtggtgcatggctgtcgtcagctcgtgtcgtgagatgttgggttaagtcccgcaacgagcgcaacccttgttgtcagttgccagcattaagttgggcactctggcgagactgccggtgacaaaccggaggaaggtggggacgacgtcaagtcatcatgccccttatgacctgggctacacacgtgctacaatggacggtacaacgagtcgcaagaccgcgaggtttagctaatctcttaaagccgttctcagttcggattgtaggctgcaactcgcctacatgaagtcggaatcgctagtaatcgcgaatcagcatgtcgcggtgaatacgttcccgggccttgtacacaccgcccgtcacaccatgagagtttgtaacacccaaagccggtggggtaaccgcaaggagccagccgtctaaggtgggacagatgattggggtgaagtcgtaacaag